(19) United States
(12) Patent Application Publication
(10) Pub. No.: US 2006/0100646 A1
(43) Pub. Date: May 11, 2006

(54) Surgical clip and method for making same

(76) Inventors: Charles C. Hart, Summerville, SC (US); Henry Kahle, Trabuco Canyon, CA (US); Ghassan Sakakine, Rancho Santa Margarita, CA (US)

Correspondence Address: APPLIED MEDICAL RESOUCES CORPORATION, 22872 Avenida Empresa, Rancho Santa Margarita, CA, 92688, US

(21) Appl. No.: 10/541,961

(22) PCT Filed: Feb. 2, 2004
(86) PCT No.: PCT/US04/02862

§ 371 Date: Jul. 11, 2005

Related U.S. Application Data
(60) Provisional application No. 60/448,022, filed on Feb. 18, 2003.

(51) Int. Cl. A61B 17/08

(52) U.S. Cl. 606/151

(57) ABSTRACT

An improved clip or clamp is provided having a one-piece wire-form construction that is suited for rapid and repeatable manufacture. The clamp, which may be disposable or reusable, includes a first jaw portion, a second jaw portion, a first handle portion, a second handle portion and a central coil or tensioning device, all of which are formed from a single piece of wire. The central coil or tensioning device comprises one or more turns. The handle portions may include ring-shaped distal ends that further enhance grip. The jaw portions may be shaped, curved, bent or otherwise configured to provide access to specific areas of a procedure. The clamp may be coated with a material providing improved traction and padding. In a method of the invention, a single wire is wound at least one turn forming the central coil and two extensions. A first extension of the wire is formed into the first jaw portion by bending back on itself, the first extension is then returned to the central coil where it is formed to rest upon an outer surface of the central coil. The bent first extension, which now extends rearward in a direction opposite to the direction of the first jaw portion, is then bent again forming the first handle portion. A second extension is similarly bent to form the second jaw portion and the second handle portion. The single-wire construction of the clamp may be achieved through programmed wire forming. In another aspect of the invention, a plurality of clamps may be placed upon a traction rod made from a metal or plastic material to align two or more stump portions of a severed vessel or tissue for reconnection or anastomosis.

This is a non-provisional application claiming the priority of provisional application Ser. No. 60/448,022, filed on Feb. 18, 2003, entitled “Surgical Clip and Method for Making Same,” which is fully incorporated herein by reference.

### BACKGROUND OF THE INVENTION

1. Field of the Invention

This invention generally relates to surgical clips and clamps and, more specifically, to a surgical clip or clamp having a one-piece wire-form construction suited for rapid and low cost manufacture.

2. Discussion of the Prior Art

Clips and clamps are commonly used in surgical procedures to interrupt or inhibit fluid flow in body conduits such as arteries, veins and ducts. These clips and clamps may be disposable or reusable. The disposable clips and clamps are typically made of plastic while the reusable clips and clamps are generally made of metal. These devices usually include extension or compression springs for biasing the opposed jaws to a closed position. In particular, these clips typically comprise a pair of opposed jaws that are closed by a spring. The opposed jaws may be shaped, curved, bent or otherwise configured to provide access to specific areas of a procedure. In addition, the jaws may be serrated or the surfaces of the jaws may be treated to provide improved traction.

Currently, it is very costly to make metal clips and clamps, especially the small clips and clamps that require great precision in the manufacturing process. Moreover, in order to reuse these clips and clamps, they have to be carefully cleaned and stored in specialized containers, the process of which is time consuming and costly. Alternatively, a plastic clip or clamp may be used in place of the metal device. The plastic clip or clamp, however, requires a different construction and a similarly sized clip or clamp does not provide the same traction or clamping force as its metal counterpart. As a consequence, larger and bulkier plastic clips or clamps have to be used in place of the metal devices. Therefore, there is a need in the art for an improved metal clip or clamp that is simple in design and construction. It is desirable that the improved clip or clamp is constructed from a single wire that is suited for rapid and repeatable manufacture. The improved clip or clamp should be disposable or reusable.

### SUMMARY OF THE INVENTION

An improved clip or clamp is provided having a one-piece wire-form construction that is suited for rapid and repeatable manufacture. The improved clamp is disposable or reusable. The improved clamp includes a first jaw portion, a second jaw portion, a first handle portion, a second handle portion and a central coil or tensioning device, all of which are formed from a single piece of wire. The central coil or tensioning device has a longitudinal axis and comprises one or more turns. The handle portions may include ring-shaped distal tips that further enhance grip. The improved clamp operates by squeezing together the handle portions about the longitudinal axis of the central coil to open the jaw portions, placing the jaw portions around a target body conduit, and slowly letting go of the handle portions to allow the jaw portions to close on the target body conduit. The clamps contemplated by the present invention may be of any size in accordance with the needs and requirements of each surgical procedure.

Different wire materials, wire diameters and coil turns may be used to provide the various tensions, strengths and other holding characteristics of the clamp. The wire material may be of a specific hardness or temper to provide specific strength and tension properties. In another aspect of the invention, a wire may be flattened or coined to provide a particular profile, shape or embossing. In addition, the jaw portions of the clamp may be shaped, curved, bent or otherwise configured to provide access to specific areas of a procedure. The clamp may also be coated with a material providing improved traction and padding. The material used for coating may be thermoset or thermoplastic materials, examples of which include soft silicone elastomer, Krayton, PVC, polyisoprene and the like. In another aspect of the invention, the entire clamp may be coated with one material and the jaw portions coated with another material.

In yet another aspect of the invention, a single wire is wound at least one turn forming the central coil and two extensions. A first extension of the wire is formed into the first jaw portion by bending back on itself, the first extension is then returned to the central coil where it is formed to rest upon an outer surface of the central coil. The bent first extension, which now extends rearward in a direction opposite to the direction of the first jaw portion, is then bent again forming the first handle portion. Similarly, a second extension of the wire is formed into the second jaw portion by bending back on itself, the second extension is then returned to the central coil where it is formed to rest upon an outer surface of the central coil. The bent second extension, which now extends rearward in a direction opposite to the direction of the second jaw portion, is then bent again forming the second handle portion.

The single-wire construction of the improved clamp may be achieved through programmed wire forming, which is highly automated and cost effective. The clamp of the invention is easy to clean and sterilize since there are no rivets or joined components.

In another aspect of the invention, a plurality of clamps may be placed upon a traction rod, which is sized and configured to fit neatly into the lumen of the central coils of the clamps. The clamps are held in place upon the traction rod by friction or by a plurality of detente features on the traction rod. The clamps placed on the traction rod may be used to align two or more stump portions of a severed vessel or tissue for reconnection or anastomosis. The traction rod is preferably made from a malleable metal or plastic material that allows a user to precisely align two or more portions of a vessel or tissue for suturing, stapling or gluing. The variety of jaw shapes of the clamps may be combined with the malleable traction rod to provide a plurality of attachment possibilities.

These and other features and advantages of the invention will become more apparent with a discussion of preferred embodiments in reference to the associated drawings.

### DESCRIPTION OF THE DRAWINGS

FIG. 1 is a perspective frontal view of the clamp of the present invention;

FIG. 2 is a perspective rear view of the clamp of the present invention;

FIG. 3 is a top view of the clamp of the present invention;

FIG. 4 is a side view of the clamp in an open condition;

FIG. 5 is a perspective view of the clamp in an open condition;

FIG. 6 is a side view of the clamp closing;

FIG. 7 illustrates a straight-jaw clamp in a closed condition;

FIG. 8 illustrates a straight-jaw clamp in an open condition;

FIG. 9 illustrates a straight-jaw clamp closing on a vessel;

FIG. 10 illustrates a curved-jaw clamp in a closed condition;

FIG. 11 illustrates a curved-jaw clamp closing on a vessel;

FIG. 12 illustrates a curved-jaw clamp closed on a vessel;

FIG. 13 is an enlarged view of the clamp showing a traction feature;

FIGS. 14-19 show a jaw portion having a curved jaw profile, an angular jaw profile, a rectangular jaw profile, a semi-circular jaw profile, a right angle jaw profile, and a circular jaw profile, respectively;

FIG. 20 illustrates a plurality of clamps used upon a traction rod;

FIGS. 21 and 22 illustrate a plurality of clamps placed upon a traction rod for anastomosis of a-vessel;

FIG. 23 illustrates the use of a shapeable traction rod with a plurality of clamps;

FIG. 24 shows a preferred wire having a round cross-section;

FIG. 25 shows a wire having a square cross-section in accordance with another embodiment of the invention;

FIG. 26 shows a wire having a rectangular cross-section in accordance with another embodiment of the invention; and

FIGS. 27(a) and 27(b) illustrate perspective views of a clamp formed of a single wire having a rectangular cross-section folded in a single plane in accordance with another embodiment of the invention.

### DESCRIPTION OF PREFERRED EMBODIMENTS AND BEST MODE OF THE INVENTION

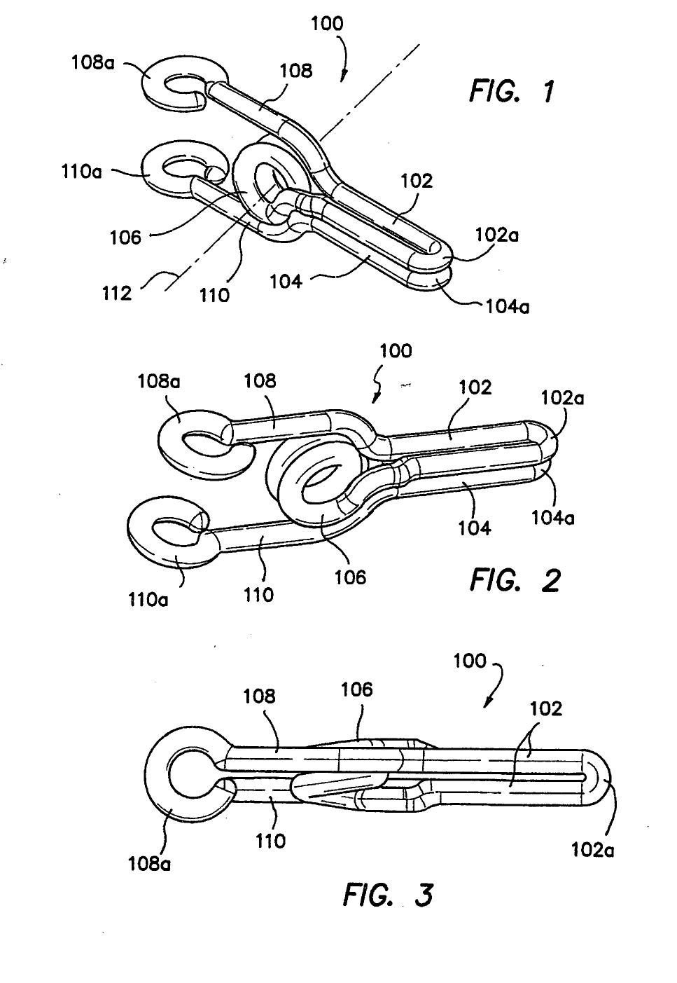

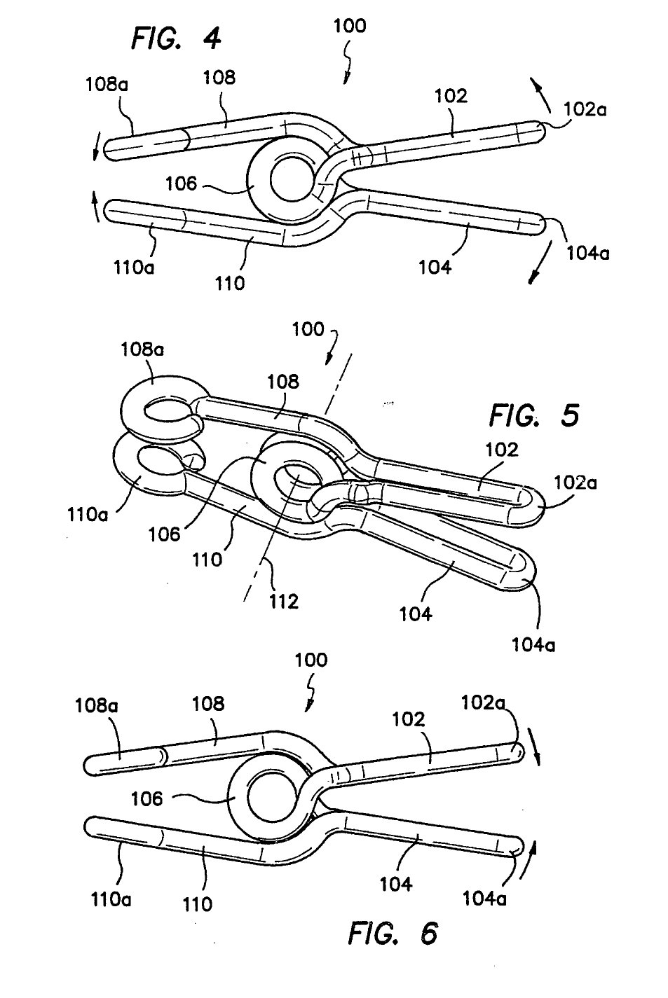

FIGS. 1-3 illustrate a surgical clamp 100 in accordance with the first embodiment of the invention. Surgical clamp 100 is unique in that it is formed from a single piece of wire and includes a first jaw portion 102, a second jaw portion 104, a first handle portion 108, a second handle portion 110 and a central coil or tensioning device 106 operatively connected to the first jaw portion 102, second jaw portion 104, first handle portion 108 and second handle portion 110. The central coil or tensioning device 106 has a longitudinal axis 112 and comprises one or more turns. The handle portions 108 and 110 may include ring-shaped distal tips 108a and 110a, respectively, that further enhance grip. The clamp 100 operates by squeezing together the handle portions 108 and 110 about the axis 112 to open the jaw portions 102 and 104, respectively, placing the jaw portions 102 and 104 around a target body conduit, and slowly letting go of the handle portions 108 and 110 to allow the jaw portions 102 and 104 to close on the target body conduit.

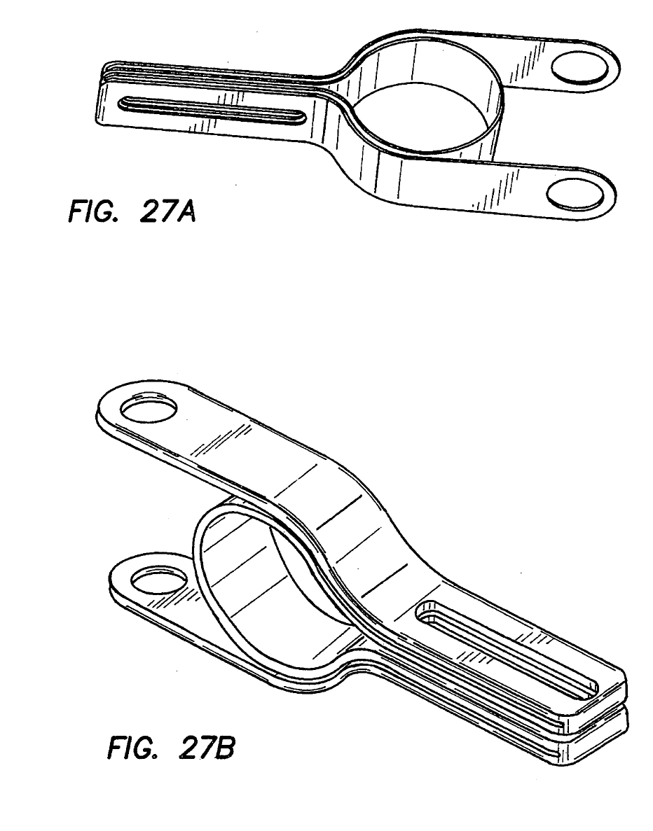

It is appreciated that various tensions, strengths and other holding characteristics may be derived from the use of different wire materials, wire diameters and coil turns. The wire material may be of a specific hardness or temper to provide specific strength and tension properties. In addition, the wire material chosen may have a specific “drawn” profile that adds specific properties to the clamp. FIGS. 24-26 illustrate that the wire may be round, square or rectangular, respectively. FIGS. 27(a) and 27(b) illustrate perspective views of a clamp formed of a single wire having a rectangular cross-section folded in a single plane. In another aspect of the invention, a round wire may be flattened or coined to provide a particular profile, shape or embossing. In this case, the clamp may be formed of a round wire and subsequently embossed by compressing the jaws over a double-sided serrated or “patterned” mandrel to impress the features of the mandrel upon the opposing jaw surfaces. In yet another aspect of the invention, the clamp may be hardened in specific areas or regions to provide specific properties to the selected areas or regions.

In accordance with a preferred method of the invention, a single wire is wound at least one turn forming the central coil or tensioning device 106 and two extensions. A first extension of the wire is formed into the first jaw portion 102 by bending back on itself, the first extension is then returned to the central coil 106 where it is formed to rest upon an outer surface of the central coil 106. The bent first extension, which now extends rearward in a direction opposite to the direction of the first jaw portion 102, is then bent again forming the first handle portion 108. Similarly, a second extension of the wire is formed into the second jaw portion 104 by bending back on itself, the second extension is then returned to the central coil 106 where it is formed to rest upon an outer surface of the central coil 106. The bent second extension, which now extends rearward in a direction opposite to the direction of the second jaw portion 104, is then bent again forming the second handle portion 110.

More specifically, the central coil 106 is formed having a first extension and a second extension. The first extension is extended for a desired length and is bent defining a first jaw length. In particular, the first extension is bent upon itself forming the first jaw portion 102. After the first jaw portion 102 is formed, the first extension is returned to the central coil 106 where it is bent around the coil 106 and continues to extend rearward in a direction opposite to the direction of the first jaw portion 102. Thus, the first jaw portion 102 comprises the first extension of the wire that is doubled by being bent back upon itself. The first jaw portion 102 has a rounded atraumatic distal end 102a and a margin between the bent wire. The returning first extension is formed to rest upon the central coil 106 and extends rearward for a first distance where it is subsequently bent forming the first handle portion 108.

Similarly, the second extension is extended for a desired length and is bent defining a second jaw length. That is, the second extension is bent upon itself forming the second jaw portion 104. After the second jaw portion 104 is formed, the second extension is returned to the central coil 106 where it is bent around the coil 106 and continues to extend rearward in a direction opposite to the direction of the second jaw portion 104. The second jaw portion 104 has a rounded atraumatic distal end 104a and a margin between the bent wire. The returning second extension is formed to rest upon the central coil 106 and extends rearward for a second distance where it is subsequently bent forming the second handle portion 110.

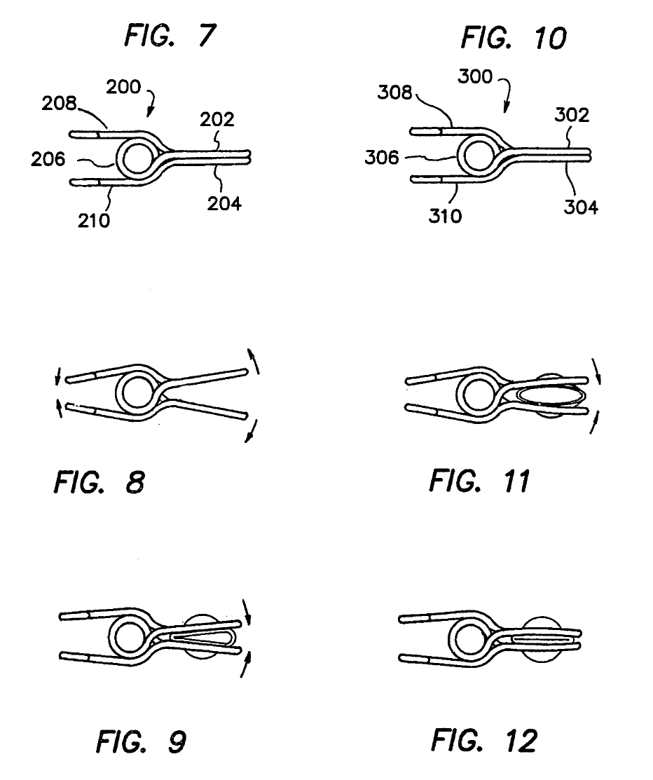

Referring to FIGS. 7-12, two jaw profiles are shown in two series of conditions that illustrate the clamps of the present invention in use. More specifically, FIGS. 7-9 illustrate a straight-jaw clamp 200 in a closed, open and closing condition, respectively. FIGS. 10-12 illustrate a curved-jaw clamp 300 in a closed, closing on a vessel, and closed on a vessel condition, respectively. The straight-jaw clamp 200 is similar to small clamps of this nature. The straight-jaw clamp 200 may be appropriate for use with very thin materials or very thin-walled vessels where the clamping force is needed toward the ultimate end of the clamp stroke. In some cases, however, a curved jaw such as the one illustrated in FIGS. 10-12 may be appropriate for use where thick materials or vessels having thick or irregular walls must be occluded. The above-described clamps 200 and 300 operate by squeezing together the handle portions to open the jaws, placing the jaws around a target body conduit, and slowly letting go of the handle portions to allow the jaws to close on the target body conduit.

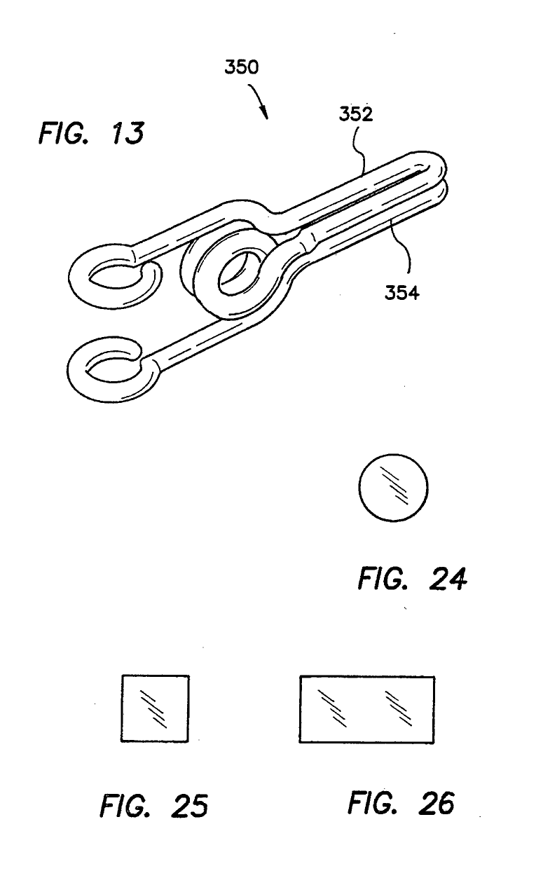

FIG. 13 illustrates an enlarged view of a clamp 350 having jaws 352 and 354 with additional traction capabilities. In particular, the jaws 352 and 354 of clamp 350 are coated with a material that enhances traction and provides a soft and atraumatic padding over the jaws. It should be noted that the margins between the two wire extensions that form the jaws already provide enhanced traction. That is, the clamp is not likely to slip along the length of a vessel due to the vessel material that is urged into the margin when the jaws are closed. Stated another way, traction is separated from the force required to occlude the vessel. As such, the jaw portions of the clamp may be coated with more than one material to enhance traction and to protect delicate body tissues. The materials used to coat or cover the jaw portions may be soft silicone elastomer, Krayton, PVC, polyisoprene and the like. In another aspect of the invention, the entire clamp may be coated with one material and the jaw portions coated with another material. A traction enhancement feature of the invention may be added to the opposing surfaces of the jaws during the application of a jaw coating by placing an embossed wafer between the jaws after they have been coated and while they cure. In yet another embodiment of the invention, disposable or reusable inserts may also be placed over the jaw portions of the clamp to improve traction.

It is appreciated that the clamps contemplated by the present invention may be of any size in accordance with the needs and requirements of each surgical procedure. However, it should be noted that the advantages of the invention are most apparent in clamps that are very small. For instance, aneurysm clamps, peripheral vascular clamps and neurological clamps may be only 0.50″ to 0.75″ in length. These clamps are very small and delicate and they tend to get lost and damaged. As such, with the ring-shaped handle construction of the clamps of the invention, a tether or suture may be attached to the handle portions to provide easy tracking, retrieval and accounting. It is appreciated, however, that the handle portions may be shaped to a variety of shapes (e.g., round, oval, triangular, rectangular, etc.) in accordance with the intended use of the clamp. The handle portion may even be a straight handle with an atraumatic tip that could be very useful in low-profile situations. A clamp having a straight handle may be applied using a special applier for placing of the clamp.

The single-wire construction of the clamp of the present invention is of particular interest. In particular, the single-wire construction may be achieved through programmed wire forming, which is highly automated and cost effective. As a result, the clamps made according to the present invention are inexpensive to produce. Moreover, the clamps of the invention are easy to clean and sterilize since there are no rivets or joined components. It is further appreciated that the inexpensive construction of the clamps of the invention also favors the single use of the clamps. As a result, the clamps of the invention may be disposable or reusable depending on a user's preference.

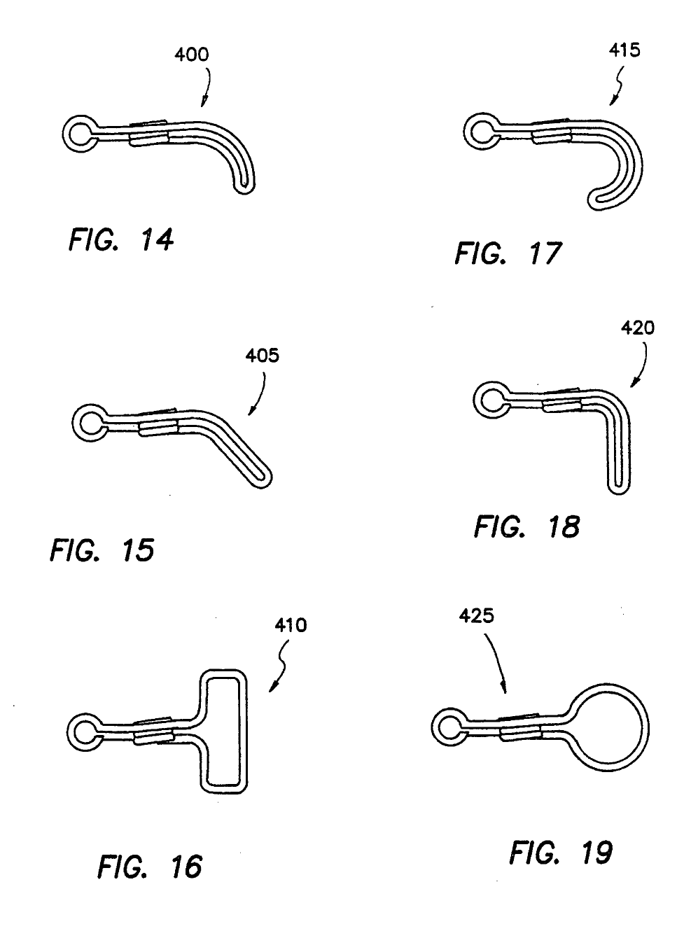

Referring to FIGS. 14-19, there are shown clamps having a curved jaw profile 400, an angular jaw profile 405, a rectangular jaw profile 410, a semi-circular jaw profile 415, a right angle jaw profile 420, and a circular jaw profile 425, respectively, in accordance with the many embodiments of the jaw portions of the invention. The jaw portions may be shaped to a variety of shapes in accordance with the intended use of the clamp. In addition to the standard shapes common to the clamps of the prior art, the jaw portions of the present invention may be formed to various open shapes that would be unattainable in clamps of the prior art. More specifically, the doubled-over wire construction of the jaw portions of the invention allow the width, length and shape of the jaws to be adjusted during manufacture or even during use. For instance, a tool may be supplied to a user to allow the user to reshape the jaw portions to a custom configuration during use. Alternatively, a clamp may be constructed such that only the central coil and the handle portions are hardened. This would leave the jaw portions in a softer condition to allow a user to form them in a desired shape.

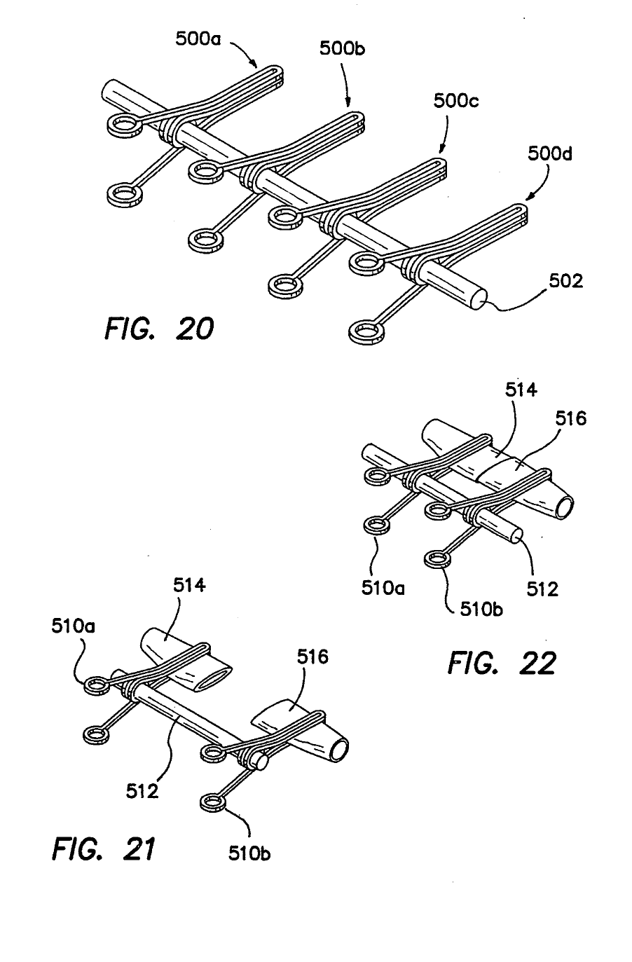

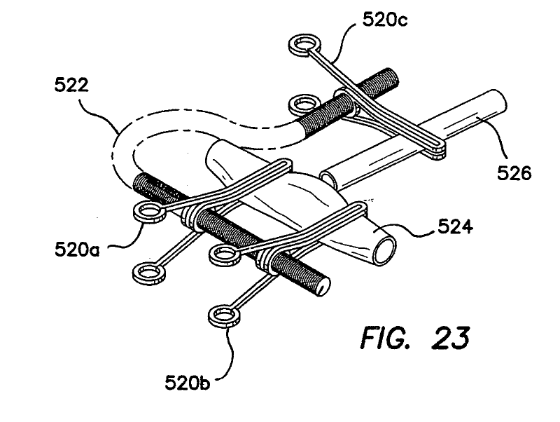

FIGS. 20-23 illustrate a plurality of clamps used upon a traction rod. As illustrated in FIG. 20, a traction rod 502 is sized and configured to fit neatly into the lumen created by the central coils of clamps 500a, 500b, 500c and 500d. The clamps 500a, 500b, 500c and 500d slide easily upon the traction rod 502 when the handles of the respective clamps are slightly depressed. This is because the central coils of the clamps slightly open when the handles are depressed. The clamps are held in place upon the traction rod by friction or by a plurality of detente features on the traction rod. In another aspect of the invention as illustrated in FIGS. 21 and 22, clamps 510a and 510b placed on a traction rod 512 may be used to align two or more stump portions 514 and 516 of a severed vessel or tissue for reconnection or anastomosis. In a preferred embodiment, the traction rod of the invention is made from a malleable metal or plastic material that allows a user to precisely align two or more portions of tissue for suturing or stapling. As illustrated in FIG. 23, it can be seen that a malleable or shapeable traction rod 522 may be bent or adjusted to align body tissues and conduits and to accommodate the position requirements of a surgical procedure. Individual clamps 520a, 520b and 520c may be placed upon the traction rod 522 to approximate tissue or vessels 524 and 526 for connection by suturing, stapling or gluing. Referring back to FIGS. 14-19, it can be seen that the variety of jaw shapes of the clamps may be combined with the malleable traction rod to provide a plurality of attachment possibilities.

Although exemplary embodiments of the invention have been shown and described, many other changes, modifications and substitutions will now be apparent to those of ordinary skill in the art, without necessarily departing from the spirit and scope of this invention.

1. A one-piece surgical clip, comprising:
a first jaw having a distal end and a proximal end;
a second jaw opposed to said first jaw having a distal end and a proximal end;
a first arm operably attached to the proximal end of the first jaw to pressably open or close said first jaw; and
a second arm operably attached to the proximal end of the second jaw to pressably open or close said second jaw,
wherein said clip is constructed from a single wire.

2. The surgical clip of claim 1, wherein the first and second arms are resiliently pressed against each other in the closed position.

3. The surgical clip of claim 1, further comprising a tensioning device operably connected to the first jaw and the second jaw to bias the first and second jaws to a closed position.

4. The surgical clip of claim 3, wherein the tensioning device is a central coil having at least one turn.

5. The surgical clip of claim 1, wherein the tension, strength and holding characteristics of the tensioning device may be varied based on the number of turns of the central coil and the hardness or temper of the wire.

6. The surgical clip of claim 1, wherein the wire has a cross-section having an oval, circular, square, rectangular or any geometric shape.

7. The surgical clip of claim 1, wherein the wire is folded in one plane.

8. The surgical clip of claim 1, wherein the first and second jaws may be curved, shaped, bent or configured to provide access to a specific area of a procedure.

9. The surgical clip of claim 1, wherein at least one of the first arm and the second arm includes a hole or a ring-shaped proximal tip to further enhance grip.

10. The surgical clip of claim 9, wherein the hole or ring-shaped proximal tip is used to attach a tether or suture to provide easy tracking, retrieval and accounting of the clip.

11. The surgical clip of claim 1, wherein each of the first and second jaws includes an atraumatic distal end.

12. The surgical clip of claim 1, wherein the first and second jaws are serrated to provide improved traction.

13. The surgical clip of claim 1, further comprising a coating over the first and second jaws to provide improved traction and padding.

14. The surgical clip of claim 13, wherein the coating includes at least one of soft elastomer, Kraton, PVC, polyisoprene and the like.

15. The surgical clip of claim 13, wherein the first and second jaws are coated with one material and the rest of the clip is coated with another material.

16. The surgical clip of claim 1, further comprising a tool to reshape the first and second jaws to a custom configuration.

17. The surgical clip of claim 3, further comprising a traction rod sized and configured to fit into a lumen of the tensioning device to place the clip at a desired position or location during a procedure.

18. The surgical clip of claim 1, wherein the clip is used as an aneurysm clamp, a peripheral vascular clamp or as a neurological clamp.

19. The surgical clip of claim 1, wherein the clip is disposable.

20. The surgical clip of claim 1, wherein the clip is reusable.

21. The surgical clip of claim 1, wherein the wire is formed from stamping.

22. The surgical clip of claim 1, wherein the wire is formed from a pre-formed ribbon of metal.

23. The surgical clip of claim 1, wherein the wire is formed from a die-cut pre-form.

24. The surgical clip of claim 1, wherein the wire is formed from at least one of a photo-chemically cut, an etched pre-form and a sheet of pre-formed ribbon.

25. The surgical clip of claim 1, wherein the wire is formed from a plurality of pre-forms generated on a single sheet.

26. A method of forming a one-piece surgical clip, comprising:
winding a single wire around a support member or mandrel at least one turn forming a central coil, a first extension and a second extension;
bending the first extension back on itself forming a first jaw and bending the second extension back on itself forming the second jaw; and
returning the first bent extension and the second bent extension to the central coil such that the first and second extensions extend rearward in a direction opposite to the direction of the first and second jaws to form a first handle portion and a second handle portion.

27. The method of claim 26, further comprising bending the proximal tips of the first and second handle portions to further enhance grip.

28. The method of claim 26, wherein each of the first and second bent extensions includes an atraumatic distal end.

29. The method of claim 26, wherein the returning first and second bent extensions are formed to rest upon the central coil.

30. The method of claim 26, wherein the first and second jaws may be curved, shaped, bent or configured to provide access to a specific area of a procedure.

31. The method of claim 26, further comprising coating the first and second jaws with at least one of an atraumatic and a traction-enhancing material.

32. The method of claim 26, wherein traction is separated from the force required to occlude a bodily tissue or vessel.

33. The method of claim 31, wherein the coating includes at least one of soft elastomer, Krayton, PVC, polyisoprene and the like.

34. The method of claim 26, further comprising coating the first and second jaws with a first traction-enhancing material and coating the rest of the clip with a second material.

35. The method of claim 26, further comprising placing disposable inserts over the first and second jaws to improve traction.

36. The method of claim 26, further comprising placing reusable inserts over the first and second jaws to improve traction.

37. The method of claim 26, wherein at least one of the first handle portion and the second handle portion includes a hole or a ring-shaped proximal tip to further enhance grip.

38. The method of claim 37, further comprising placing a tether or suture through one of the handle portions to provide easy tracking, retrieval and accounting of the clip.

39. The method of claim 26, wherein the process is automated.

40. The method of claim 26, wherein the wire has a cross-section having an oval, circular, square, rectangular or any geometric shape.

41. A one-piece surgical clip, comprising:
a first jaw having a distal end and a proximal end;
a second jaw opposed to said first jaw having a distal end and a proximal end;
a first arm integrally formed at the proximal end of the first jaw to pressably open or close said first jaw; and
a second arm integrally formed at the proximal end of the second jaw to pressably open or close said second jaw,
wherein said clip is constructed from a single wire

42. The surgical clip of claim 40, further comprising a tensioning device integrally formed with the first jaw and the second jaw to bias the first and second jaws to a closed position.
